(19) United States
(12) Patent Application Publication
(10) Pub. No.: US 2026/0124609 A1
(43) Pub. Date: May 7, 2026

(54) PREPARATION METHOD OF DUAL-ANION CATALYST, AND USE OF DUAL-ANION CATALYST IN CATALYTIC DEGRADATION OF POLYURETHANE MATERIAL

(71) Applicant: ZHEJIANG UNIVERSITY OF TECHNOLOGY, Hangzhou (CN)

(72) Inventors: Huiwen HE, Huzhou (CN); Kaiming DU, Huzhou (CN); Xu WANG, Huzhou (CN); Yulu ZHU, Huzhou (CN); Si CHEN, Huzhou (CN); Meng MA, Huzhou (CN); Yanqin SHI, Huzhou (CN)

(73) Assignee: ZHEJIANG UNIVERSITY OF TECHNOLOGY, Hangzhou (CN)

(21) Appl. No.: 19/437,378

(22) Filed: Dec. 31, 2025

Related U.S. Application Data
(63) Continuation of application No. PCT/CN2024/126578, filed on Oct. 22, 2024.

(30) Foreign Application Priority Data
Aug. 27, 2024 (CN) 202411183355.9

(51) Int. Cl. B01J 31/18; B01J 31/22; B01J 37/00; B01J 37/08; C07C 263/04; C08J 11/28

(52) U.S. Cl. CPC B01J 31/181; B01J 31/2226; B01J 37/009; B01J 37/08; C07C 263/04; C08J 11/28; B01J 2231/005; B01J 2531/22; B01J 2531/26; B01J 2531/847; C08J 2375/04

(57) ABSTRACT

A preparation method of a dual-anion catalyst, and a use of the dual-anion catalyst in catalytic degradation of a polyurethane material are provided. The preparation method includes: dissolving a metal salt in deionized water to produce a metal salt solution; adding a nitrogen-containing heterocyclic compound with hydroxyl or amino as a substrate to a reactor, adding the metal salt solution dropwise to the reactor, and conducting condensation reflux at 60° C. to 150° C. for 1 h to 8 h; adding an amine compound dropwise, and conducting a reaction; after the reaction is completed, conducting vacuum filtration to produce a filtrate; and subjecting the filtrate to evaporation, and oven-drying a resulting product to produce the dual-anion catalyst. A recovered polyol obtained through the degradation with the catalyst exhibits basically the same fundamental physical properties such as hydroxyl value, amine value, and viscosity to a virgin polyol.

### CROSS REFERENCE TO THE RELATED APPLICATIONS

This application is a continuation application of International Application No. PCT/CN2024/126578, filed on Oct. 22, 2024, which is based upon and claims priority to Chinese Patent Application No. 202411183355.9, filed on Aug. 27, 2024, the entire contents of which are incorporated herein by reference.

### TECHNICAL FIELD

The present disclosure relates to the field of chemical catalytic degradation and recycling of waste polyurethane materials, and specifically relates to a preparation method of a dual-anion catalyst, and a use of the dual-anion catalyst in catalytic degradation of a polyurethane material.

### BACKGROUND

The recycling and reuse of waste polymer materials is a crucial pathway for enabling a green circular economy and achieving the goals of peak carbon dioxide emissions and carbon neutrality. The annual consumption of polyurethane products in China exceeds 15 million tons, with waste production reaching 400,000 tons per year. Polyurethane materials are widely used in aerospace, household goods, transportation, and other fields. However, polyurethane materials can hardly be recycled due to insoluble and non-melting cross-linked structures. Waste polyurethane can only be disposed of through landfill or incineration, or can only be processed into low-end products through crushing and compaction, with a recycling rate of less than 10%. This leads to severe ecological and environmental problems and significant resource waste. Additionally, common polyurethane materials are often mixtures characterized by complex structures and variable compositions, which pose significant challenges for chemical recycling.

Currently, the chemical depolymerization methods for waste polyurethane primarily include alcoholysis, acidolysis, aminolysis, hydrolysis, and enzymolysis. The common goal of these methods is to depolymerize polyurethane into polymerizable polyols, thereby achieving the recycling of resources and the treatment of non-depolymerizable wastes. However, for high-volume flexible polyurethane foam that demands high-quality recycling, there is still a lack of efficient depolymerization and recycling technologies. The invention patent CN201910863314.7 discloses a method for preparing a polyether polyol through hydrothermal catalytic degradation of a waste rigid polyurethane material. In this method, the polyether polyol is prepared by a catalytic solvothermal technology. A catalyst used in this method does not need to be recovered, but can remain in a reaction product to further serve as a catalyst for synthesizing a polyurethane material. However, this method requires a relatively high temperature (180° C. to 240° C.). Most importantly, this method is limited to rigid polyurethane foam. The invention patent CN201710649420.6 discloses a method for acidolysis of a flexible polyurethane foam waste to produce a polyol. In this method, a solid acid catalyst, a liquid acid catalyst, an impregnated acid catalyst, a cation exchange resin, or a metal-salt acid catalyst is used to achieve the degradation of the flexible polyurethane foam waste. However, this method requires a reaction time of 9 h or more and a relatively high reaction temperature (230° C. to 250° C.). Moreover, the catalysts mentioned above cannot significantly enhance the reaction rate or reduce the reaction activation energy.

In summary, to achieve the eco-friendly recovery of high-quality polyols, it is essential to clarify a degradation mechanism of polyurethane, develop a key degradation catalyst for polyurethane, and activate urethane bonds at the source by a catalytic technology to enhance the attack capability of a nucleophilic reagent. Consequently, the reaction temperature can be lowered, the generation of harmful by-products such as aromatic amines can be avoided, the reaction efficiency can be improved, and the quality of a recovered polyol can be enhanced.

### SUMMARY

The present disclosure is intended to provide a preparation method of a dual-anion catalyst, and a use of the dual-anion catalyst in catalytic degradation of a polyurethane material. This catalyst plays a catalytic role through a synergistic action in the following two aspects: In a first aspect, an amine compound enhances the attack capability of a nucleophilic reagent itself. In a second aspect, a complex formed by a metal ion and a nitrogen-containing heterocyclic compound boosts the electropositivity of carbonyl carbon of a urethane bond to make it susceptible to attack by a nucleophilic reagent, thereby enhancing the degradation rate and degree. A recovered polyol obtained through the degradation with this catalyst exhibits basically the same fundamental physical properties such as hydroxyl value, amine value, and viscosity to a virgin polyol, and can partially replace the virgin polyol in the production of a flexible polyurethane foam.

Technical solutions of the present disclosure are as follows:

A preparation method of a dual-anion catalyst is provided, including the following steps:
1) dissolving a metal salt in deionized water to produce a metal salt solution;
2) adding a nitrogen-containing heterocyclic compound with hydroxyl or amino as a substrate to a reactor, adding the metal salt solution dropwise to the reactor, and conducting condensation reflux at 60° C. to 150° C. for 1 h to 8 h;
3) after the condensation reflux in the step 2) is completed, adding an amine compound dropwise to the reactor, and conducting a reaction for 2 h to 5 h; and after the reaction is completed, conducting vacuum filtration to produce a filtrate;
4) subjecting the filtrate obtained in the step 3) to evaporation; and
5) oven-drying a product obtained in the step 4) to produce the dual-anion catalyst.

In the step 1), the metal salt is one or more of zinc acetate, magnesium acetate, zinc nitrate, cadmium nitrate, nickel nitrate, and cobalt nitrate.

In the step 2), the nitrogen-containing heterocyclic compound with hydroxyl or amino is one or more of 4-(hydroxymethyl)imidazole, 2-hydroxymethyl-1-methylimidazole, 6-(hydroxymethyl)pyridin-3-ol, 3,4-bis(hydroxymethyl)furan, and 6-hydroxymethylquinoline.

In the step 2), the metal salt solution is slowly added dropwise to the reactor for 20 min to 40 min.

In the step 2), the condensation reflux is conducted at 100° C. to 150° C. for 1 h to 4 h.

In the step 3), the amine compound is one or more of phenylethylamine, triphenylguanidine, tetramethylguanidine, and sulfaguanidine.

In the step 3), after the condensation reflux in the step 2) is completed, the amine compound is added dropwise to the reactor, and then the reaction is conducted for 2 h to 3 h. After the reaction is completed, the vacuum filtration is conducted to produce the filtrate.

A molar ratio of the nitrogen-containing heterocyclic compound with hydroxyl or amino, the metal salt, and the amine compound is (0.3-1):(0.3-1.5):(0.5-1).

In the step 4), the filtrate obtained in the step 3) is treated for 1 h to 2 h in a rotary evaporator at 50° C. to 60° C.; and

in the step 5), the product obtained in the step 4) is oven-dried for 12 h to 24 h in a forced air oven at 80° C. to 90° C.

Specifically, a dual-anion catalyst for catalyzing the degradation of a polyurethane material is synthesized by the following process:

A specified amount of a metal salt is dissolved in deionized water to produce a metal salt solution for later use. A specified amount of a nitrogen-containing heterocyclic compound with hydroxyl or amino is added as a substrate to a three-necked flask and heated to 60° C. to 150° C., the metal salt solution prepared previously is slowly added dropwise through a constant-pressure dropping funnel, and condensation reflux is conducted for 1 h to 8 h. A specified amount of an amine compound is slowly added dropwise, and then a reaction is conducted for 2 h to 5 h. After the reaction is completed, vacuum filtration is conducted to produce a filtrate. The filtrate is treated for 1 h to 2 h in a rotary evaporator at 50° C. to 60° C. to remove water and unreacted compounds. A resulting sample is oven-dried for 12 h to 24 h in a forced air oven at 80° C. to 90° C. to produce the dual-anion catalyst.

The dual-anion catalyst for catalyzing degradation of a polyurethane material is used in a degradation process. The degradation process is a multi-stage degradation process disclosed in the previously authorized patents (patent ZL202110931533.1: “Method for Efficient and Controlled Degradation and Recycling of Polyurethane Foam to Produce Polyether Polyol”; and patent ZL202211054512.7: “Method and Device for Degradation and Recycling of Waste Polyurethane Foam”).

The degradation process is specifically as follows: 10 g of diethanolamine, 50 g of a polyether polyol, and 3 g of the dual-anion catalyst are added to a 500 mL three-necked flask. Then, a mechanical stirrer is started, and preheating is conducted at a low rotational speed and a temperature of 140° C. to 180° C. for 15 min. A rotational speed of the mechanical stirrer is increased, and 100 g of waste polyurethane foam fragments are continuously fed into the three-necked flask. After the waste polyurethane foam fragments are completely consumed, 50 g of the polyether polyol is further added, and continuous stirring is conducted for 1 h to 3 h. Then, 20 g of succinic acid is added, the rotational speed of the mechanical stirrer is further increased, and continuous stirring is further conducted for 1 h to 3 h until fundamental physical properties of a degradation product do not change significantly.

The catalyst in the present disclosure is not limited to degradation in the multi-stage degradation process, and can also bring beneficial effects in common methods such as acidolysis, alcoholysis, and aminolysis.

A synthesis theory of the present disclosure is based on the easy coordination of a metal ion with hydroxyl and the easy formation of a complex of amino with a metal ion. Therefore, the catalyst of the present disclosure is a catalyst system with a metal ion centered and coordinated or complexed with dual anions that is produced through the coordination or complexation of the metal ion with multiple nitrogen-containing heterocyclic compound molecules and an amine compound molecule.

A degradation mechanism for waste polyurethane involves the attack of a positively charged carbonyl carbon cation of a urethane bond with a nucleophilic reagent, as shown in reaction equation (1).

A mechanism for the catalytic degradation of waste polyurethane in the present disclosure is as follows:
a. A complex is generated through the complexation of an amine compound with a metal ion M⁺. At a specified temperature, a complexation bond can be cleaved to generate free AZ-O-M⁺ and R—NH₂. On one hand, R—NH₂ can provide a basic environment to enhance the ability of a nucleophilic reagent to attack carbonyl carbon of a urethane bond. On the other hand, R—NH₂ itself can act as a nucleophilic reagent capable of further attacking the carbonyl carbon of the urethane bond.
b. Free AZ-O-M⁺ represents a complex formed through the coordination of a metal ion with a nitrogen-containing heterocyclic compound. M⁺ serves as a catalytic center in a reaction. M⁺ attracts hydroxyl or amino of a nucleophilic reagent (degrading agent) and carbonyl oxygen of urethane, such that both the nucleophilic reagent (degrading agent) and an urethane bond bind to M⁺ and approach each other. As a result, an effective concentration of a substrate significantly increases to make an intermolecular reaction resemble an intramolecular reaction, thereby accelerating the reaction. M⁺ can also attract electrons of carbonyl to enhance the electropositivity of carbonyl carbon and make it susceptible to attack by the nucleophilic reagent (degrading agent). Moreover, AZ in the complex exhibits a strong electron-withdrawing ability, and can remarkably enhance the catalytic effect of M⁺.

Through a synergistic action in the above two aspects, the catalyst designed in the present disclosure not only enhances the attack capability of the nucleophilic reagent itself, but also increases the electropositivity of carbonyl carbon of the urethane bond to make it susceptible to attack by the nucleophilic reagent, as shown in reaction equation (2).

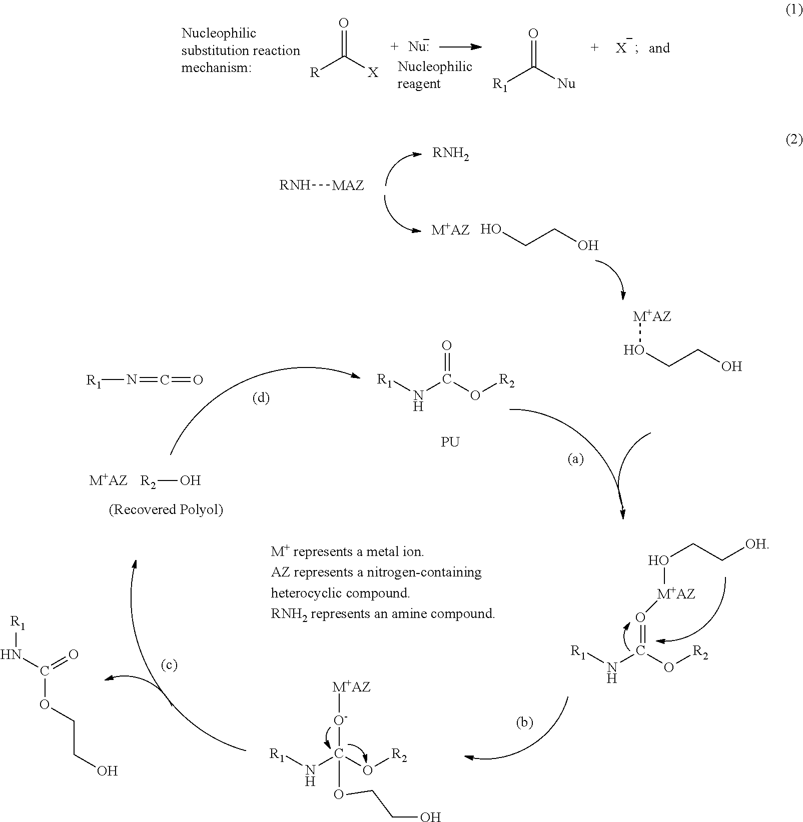

Compared with the Prior Art, the Present Disclosure has the Following Advantages:
(1) Through a synergistic effect of varying components, the catalyst of the present disclosure can significantly enhance the degradation rate and degree during a degradation process of waste polyurethane.
(2) The catalyst of the present disclosure can be prepared by a simple process, exhibits excellent activity and high-temperature stability, and holds a promising industrial application prospect.

### BRIEF DESCRIPTION OF THE DRAWINGS

FIG. 1 shows a synthesis equation of ZnA₂ and a proton nuclear magnetic resonance (¹H NMR) spectrum of a product in the present disclosure;

FIG. 2 shows a structural formula of ZnA₂;

FIG. 3 shows rebound rate data of polyurethane foams;

FIG. 4 shows 50% permanent compression set rate data of polyurethane foams; and

FIG. 5 shows 75% permanent compression set rate data of polyurethane foams.

### DETAILED DESCRIPTION OF THE EMBODIMENTS

The present disclosure is further illustrated below through specific examples, but the protection scope of the present disclosure is not limited thereto.

Unless otherwise specified, all raw materials and devices used in the present disclosure are commercially available or are commonly used in the art. Unless otherwise specified, the methods in the examples are all conventional methods in the art.

### Example 1

1.2 mol of magnesium acetate was dissolved in deionized water to prepare a magnesium acetate solution for later use. 1 mol of 3,4-bis(hydroxymethyl)furan was added as a substrate to a 500 mL three-necked flask, and condensation reflux was conducted at 150° C. The magnesium acetate solution was slowly added dropwise through a constant-pressure dropping funnel, and then a reaction was conducted for 1 h. 0.6 mol of triphenylguanidine was slowly added dropwise, and then a reaction was conducted for 3 h. Vacuum filtration was conducted to remove solid impurities. Evaporation was conducted for 2 h in a rotary evaporator at 50° C. to remove water and unreacted compounds. A resulting sample was oven-dried for 18 h in a forced air oven at 80° C. to produce a final product, Example 1.

10 g of diethanolamine, 50 g of a polyether polyol, and 3 g of Example 1 were added to a 500 mL three-necked flask. Then, a mechanical stirrer was started, and preheating was conducted at a low rotational speed and a temperature of 180° C. for 15 min. A rotational speed of the mechanical stirrer was increased, and 100 g of waste polyurethane foam fragments were continuously fed into the three-necked flask. After the waste polyurethane foam fragments were completely consumed, 50 g of the polyether polyol was further added, and continuous stirring was conducted for 2 h. Then, 20 g of succinic acid was added, the rotational speed of the mechanical stirrer was further increased, and continuous stirring was further conducted for 2 h until fundamental physical properties of a degradation product did not change significantly. The fundamental physical properties were as follows: hydroxyl value: 93 mg KOH/g, acid value: 0.6 mg KOH/g, and viscosity: 1,970 mPa·s (these physical properties were tested according to national standards).

### Example 2

1.3 mol of nickel nitrate was dissolved in deionized water to prepare a nickel nitrate solution for later use. 0.7 mol of 4-(hydroxymethyl)imidazole was added as a substrate to a 500 mL three-necked flask, and condensation reflux was conducted at 140° C. The nickel nitrate solution was slowly added dropwise through a constant-pressure dropping funnel, and then a reaction was conducted for 2 h. 0.9 mol of phenylethylamine was slowly added dropwise, and then a reaction was conducted for 2 h. Vacuum filtration was conducted to remove solid impurities. Evaporation was conducted for 1 h in a rotary evaporator at 60° C. to remove water and unreacted compounds. A resulting sample was oven-dried for 12 h in a forced air oven at 90° C. to produce a final product, Example 2.

10 g of diethanolamine, 50 g of a polyether polyol, and 3 g of Example 2 were added to a 500 mL three-necked flask. Then, a mechanical stirrer was started, and preheating was conducted at a low rotational speed and a temperature of 180° C. for 15 min. A rotational speed of the mechanical stirrer was increased, and 100 g of waste polyurethane foam fragments were continuously fed into the three-necked flask. After the waste polyurethane foam fragments were completely consumed, 50 g of the polyether polyol was further added, and continuous stirring was conducted for 2 h. Then, 20 g of succinic acid was added, the rotational speed of the mechanical stirrer was further increased, and continuous stirring was further conducted for 2 h until fundamental physical properties of a degradation product did not change significantly. The fundamental physical properties were as follows: hydroxyl value: 87 mg KOH/g, acid value: 0.6 mg KOH/g, and viscosity: 2,130 mPa·s (these physical properties were tested according to national standards).

### Example 3

0.5 mol of zinc nitrate was dissolved in deionized water to prepare a zinc nitrate solution for later use. 0.4 mol of 6-hydroxymethylquinoline was added as a substrate to a 500 mL three-necked flask, and condensation reflux was conducted at 120° C. The zinc nitrate solution was slowly added dropwise through a constant-pressure dropping funnel, and then a reaction was conducted for 1 h. 1 mol of sulfaguanidine was slowly added dropwise, and then a reaction was conducted for 3 h. Vacuum filtration was conducted to remove solid impurities. Evaporation was conducted for 1 h in a rotary evaporator at 60° C. to remove water and unreacted compounds. A resulting sample was oven-dried for 24 h in a forced air oven at 80° C. to produce a final product, Example 3.

10 g of diethanolamine, 50 g of a polyether polyol, and 3 g of Example 3 were added to a 500 mL three-necked flask. Then, a mechanical stirrer was started, and preheating was conducted at a low rotational speed and a temperature of 180° C. for 15 min. A rotational speed of the mechanical stirrer was increased, and 100 g of waste polyurethane foam fragments were continuously fed into the three-necked flask. After the waste polyurethane foam fragments were completely consumed, 50 g of the polyether polyol was further added, and continuous stirring was conducted for 2 h. Then, 20 g of succinic acid was added, the rotational speed of the mechanical stirrer was further increased, and continuous stirring was further conducted for 2 h until fundamental physical properties of a degradation product did not change significantly. The fundamental physical properties were as follows: hydroxyl value: 76 mg KOH/g, acid value: 0.6 mg KOH/g, and viscosity: 4,530 mPa·s (these physical properties were tested according to national standards).

### Example 4

1.2 mol of zinc acetate was dissolved in deionized water to prepare a zinc acetate solution for later use. 1 mol of 2-hydroxymethyl-1-methylimidazole was added as a substrate to a 500 mL three-necked flask, and condensation reflux was conducted at 150° C. The zinc acetate solution was slowly added dropwise through a constant-pressure dropping funnel, and then a reaction was conducted for 1 h. 1 mol of tetramethylguanidine was slowly added dropwise, and then a reaction was conducted for 3 h. Vacuum filtration was conducted to remove solid impurities. Evaporation was conducted for 2 h in a rotary evaporator at 50° C. to remove water and unreacted compounds. A resulting sample was oven-dried for 18 h in a forced air oven at 80° C. to produce a final product, Example 4.

10 g of diethanolamine, 50 g of a polyether polyol, and 3 g of Example 4 were added to a 500 mL three-necked flask. Then, a mechanical stirrer was started, and preheating was conducted at a low rotational speed and a temperature of 180° C. for 15 min. A rotational speed of the mechanical stirrer was increased, and 100 g of waste polyurethane foam fragments were continuously fed into the three-necked flask. After the waste polyurethane foam fragments were completely consumed, 50 g of the polyether polyol was further added, and continuous stirring was conducted for 2 h. Then, 20 g of succinic acid was added, the rotational speed of the mechanical stirrer was further increased, and continuous stirring was further conducted for 2 h until fundamental physical properties of a degradation product did not change significantly. The fundamental physical properties were as follows: hydroxyl value: 65 mg KOH/g, acid value: 0.6 mg KOH/g, and viscosity: 1,160 mPa·s (these physical properties were tested according to national standards).

TABLE 1
Performance test results for the products in the examples

| | Hydroxyl value | Acid value | Viscosity |
|---|---|---|---|
| | of a degrada- | of a degrada- | of a degrada- |
| | tion product | tion product | tion product |
| Example | (mg KOH/g) | (mg KOH/g) | (mPa · s) |
| Example 1 | 93 | 0.6 | 1970 |
| Example 2 | 87 | 0.9 | 2130 |
| Example 3 | 76 | 1.2 | 4530 |
| Example 4 | 65 | 0.2 | 1160 |

According to the comparison of physical properties of the recovered products in Examples 1 to 4 (as shown in Table 1), with a same degradation formulation, the primary factors affecting the hydroxyl value and acid value are a type and a content of the amine compound in the catalyst. According to the viscosity data of the recovered products, Example 4 demonstrates the optimal effect. To well illustrate a synergistic effect in the dual-anion catalyst, the formulation in Example 4 is selected for further investigation as follows:

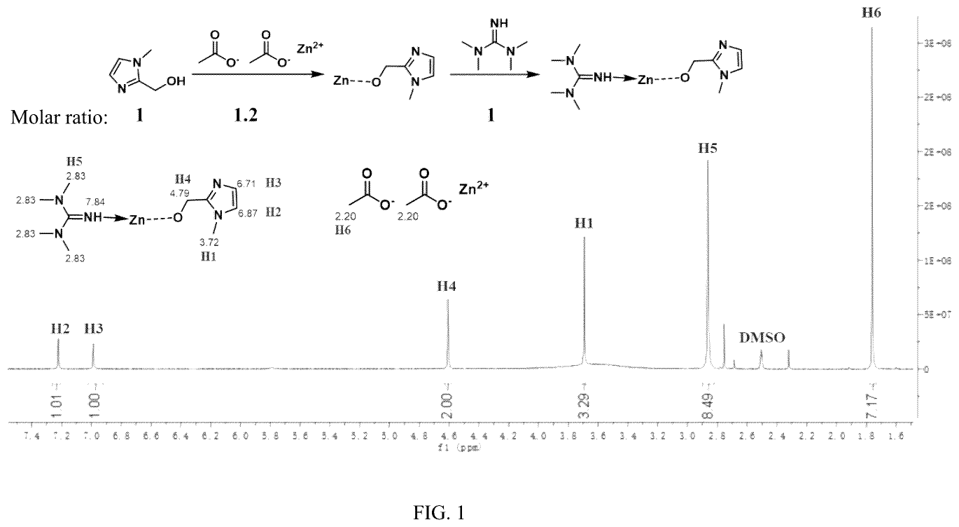

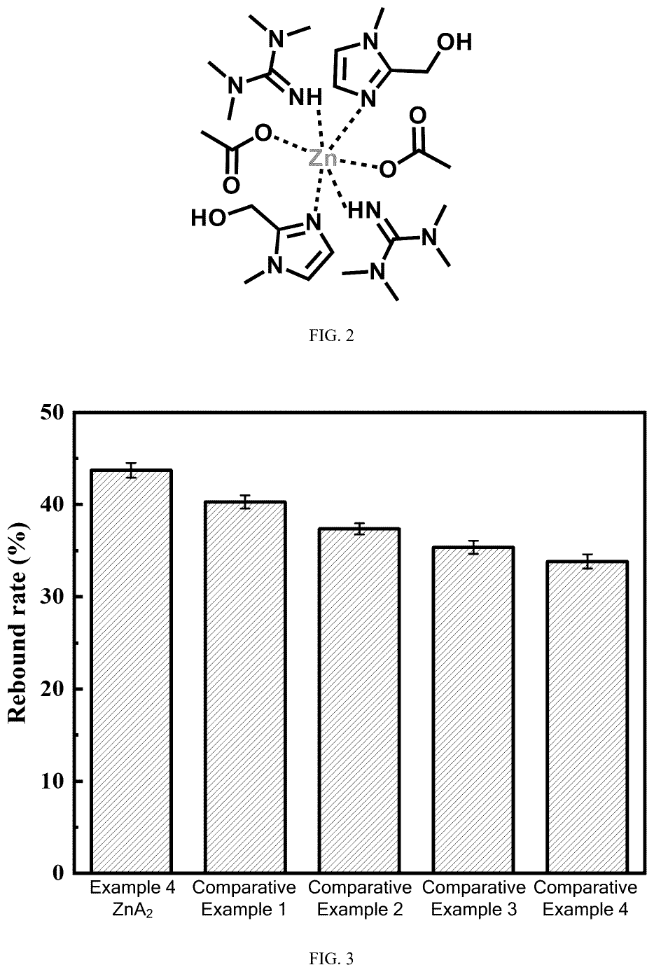

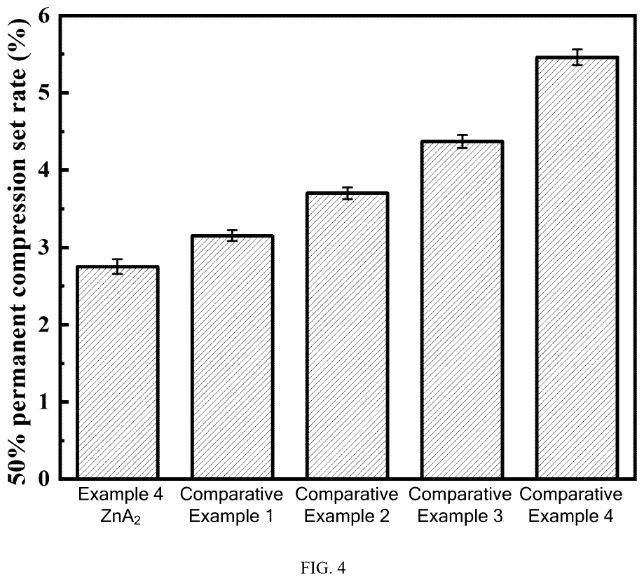

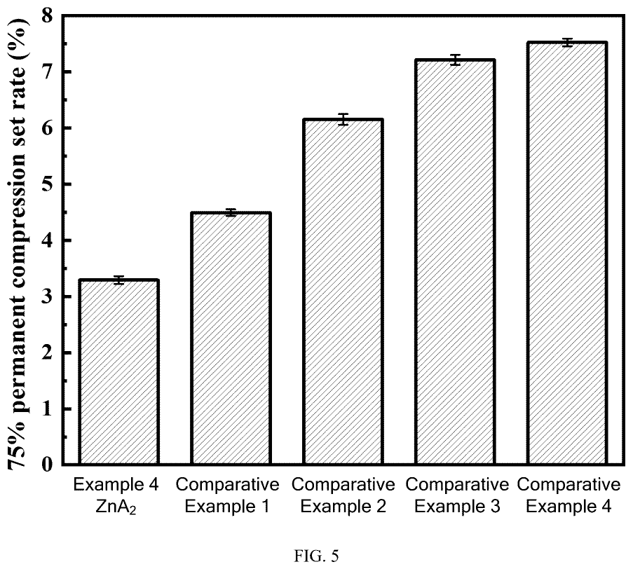

The dual-anion catalyst Example 4 obtained in Example 4 (denoted as ZnA₂) was further analyzed. According to the ¹H NMR spectrum in FIG. 1, this structure was successfully analyzed. According to inductively coupled plasma mass spectrometry (ICP-MS) test results in Table 2, a content of Zn in this product was 9.37%. A structural formula of the product inferred accordingly was shown in FIG. 2.

TABLE 2
ICP-MS test results of ZnA₂

| | Zn 66/66 | Sample | Final | | |
|---|---|---|---|---|---|
| | Ammonia DRC | weight | volume | Dilution | Zn content |
| Sample ID | (μg/L) | (g) | (mL) | factor | in a sample |
| Example 4 | 84.45568062 | 0.4470 | 50 | 10000 | 9.37% |

To demonstrate the synergistic effect in the dual-anion catalyst, the three components of this formulation were used as a catalyst separately for comparison under same experimental conditions. A specific plan was as follows:

### Comparative Example 1

10 g of diethanolamine, 50 g of a polyether polyol, and 3 g of zinc acetate were added to a 500 mL three-necked flask. Then, a mechanical stirrer was started, and preheating was conducted at a low rotational speed and a temperature of 180° C. for 15 min. A rotational speed of the mechanical stirrer was increased, and 100 g of waste polyurethane foam fragments were continuously fed into the three-necked flask. After the waste polyurethane foam fragments were completely consumed, 50 g of the polyether polyol was further added, and continuous stirring was conducted for 2 h. Then, 20 g of succinic acid was added, the rotational speed of the mechanical stirrer was further increased, and continuous stirring was further conducted for 2 h until fundamental physical properties of a degradation product did not change significantly. The fundamental physical properties were as follows: hydroxyl value: 65 mg KOH/g, acid value: 0.7 mg KOH/g, and viscosity: 3,770 mPa·s (these physical properties were tested according to national standards).

### Comparative Example 2

10 g of diethanolamine, 50 g of a polyether polyol, and 3 g of 2-hydroxymethyl-1-methylimidazole were added to a 500 mL three-necked flask. Then, a mechanical stirrer was started, and preheating was conducted at a low rotational speed and a temperature of 180° C. for 15 min. A rotational speed of the mechanical stirrer was increased, and 100 g of waste polyurethane foam fragments were continuously fed into the three-necked flask. After the waste polyurethane foam fragments were completely consumed, 50 g of the polyether polyol was further added, and continuous stirring was conducted for 2 h. Then, 20 g of succinic acid was added, the rotational speed of the mechanical stirrer was further increased, and continuous stirring was further conducted for 2 h until fundamental physical properties of a degradation product did not change significantly. The fundamental physical properties were as follows: hydroxyl value: 59 mg KOH/g, acid value: 1.3 mg KOH/g, and viscosity: 5,560 mPa·s (these physical properties were tested according to national standards).

### Comparative Example 3

10 g of diethanolamine, 50 g of a polyether polyol, and 3 g of tetramethylguanidine were added to a 500 mL three-necked flask. Then, a mechanical stirrer was started, and preheating was conducted at a low rotational speed and a temperature of 180° C. for 15 min. A rotational speed of the mechanical stirrer was increased, and 100 g of waste polyurethane foam fragments were continuously fed into the three-necked flask. After the waste polyurethane foam fragments were completely consumed, 50 g of the polyether polyol was further added, and continuous stirring was conducted for 2 h. Then, 20 g of succinic acid was added, the rotational speed of the mechanical stirrer was further increased, and continuous stirring was further conducted for 2 h until fundamental physical properties of a degradation product did not change significantly. The fundamental physical properties were as follows: hydroxyl value: 71 mg KOH/g, acid value: 1.5 mg KOH/g, and viscosity: 7,890 mPa·s (these physical properties were tested according to national standards).

### Comparative Example 4

10 g of diethanolamine and 50 g of a polyether polyol were added to a 500 mL three-necked flask. Then, a mechanical stirrer was started, and preheating was conducted at a low rotational speed and a temperature of 180° C. for 15 min. A rotational speed of the mechanical stirrer was increased, and 100 g of waste polyurethane foam fragments were continuously fed into the three-necked flask. After the waste polyurethane foam fragments were completely consumed, 50 g of the polyether polyol was further added, and continuous stirring was conducted for 2 h. Then, 20 g of succinic acid was added, the rotational speed of the mechanical stirrer was further increased, and continuous stirring was further conducted for 2 h until fundamental physical properties of a degradation product did not change significantly. The fundamental physical properties were as follows: hydroxyl value: 53 mg KOH/g, acid value: 1.5 mg KOH/g, and viscosity: 8,900 mPa·s (these physical properties were tested according to national standards).

TABLE 3
Performance test results for the products
in the example and the comparative examples

| | Hydroxyl value | Acid value | Viscosity |
|---|---|---|---|
| | of a degrada- | of a degrada- | of a degrada- |
| | tion product | tion product | tion product |
| | (mg KOH/g) | (mg KOH/g) | (mPa · s) |
| Example 4 | 65 | 0.6 | 1160 |
| (ZnA₂) | | | |
| Comparative | 65 | 0.7 | 3770 |
| Example 1 | | | |
| Comparative | 59 | 1.3 | 5560 |
| Example 2 | | | |
| Comparative | 71 | 1.5 | 7890 |
| Example 3 | | | |
| Comparative | 53 | 1.5 | 8900 |
| Example 4 | | | |

The degradation products (recovered polyols) of the example and Comparative Examples 1 to 4 were each used to replace 30% of a virgin polyol in the further preparation of a flexible polyurethane foam. At room temperature of 25° C., foaming was conducted with a polyurethane foaming formulation (Table 4). A polyether polyol, a recovered polyol, and additives other than toluene diisocyanate (TDI) were mixed with a high-speed stirrer at a rotational speed of 9,000 r/min. At the same rotational speed, TDI was immediately added, and mixing was conducted for 10 s to 15 s. A resulting mixture was poured into a mold and subjected to foaming to produce a polyurethane foam. The polyurethane foam prepared was subjected to mechanical property tests, including rebound rate, 50% permanent compression set rate, and 75% permanent compression set rate tests. Mechanical properties of the regenerated foam were shown in Table 5.

TABLE 4
Polyurethane foaming formulation

| Raw material | wt/wt |
|---|---|
| Polyether | 70 |
| polyol 5623 | |
| Recovered polyol | 30 |
| TDI | 35-45 |
| Water | 2-4 |
| Stannous octoate | 0.1-0.3 |
| Triethylenediamine | 0.1-0.2 |
| Silicone oil | 1.0-2.0 |
| Dichloromethane | 0.1-1.5 |
| Anti-aging agent | 0.1-0.2 |
| Additive | 1.0 |

TABLE 5
Mechanical properties of the regenerated foam

| | Example 4 | Comparative | Comparative | Comparative | Comparative | Performance |
|---|---|---|---|---|---|---|
| Sample | (ZnA₂) | Example 1 | Example 2 | Example 3 | Example 4 | requirement |
| Density | 28.69 | 28.30 | 28.07 | 28.07 | 28.57 | 26.6-30.8 |
| (kg/m³) | | | | | | |
| Rebound | 43.72 | 40.29 | 37.36 | 35.36 | 33.83 | ≥37% |
| rate (%) | | | | | | |
| 25% | 127 | 137 | 144 | 152 | 171 | |
| indentation | | | | | | |
| hardness (N) | | | | | | |
| 40% | 162 | 177 | 186 | 199 | 221 | 123-177 |
| indentation | | | | | | |
| hardness (N) | | | | | | |
| 65% | 322 | 362 | 361 | 385 | 403 | |
| indentation | | | | | | |
| hardness (N) | | | | | | |
| 50% | 2.75 | 3.15 | 3.7 | 4.37 | 5.46 | ≤7% |
| permanent | | | | | | |
| compression | | | | | | |
| set rate (%) | | | | | | |
| 75% | 3.29 | 4.49 | 6.15 | 7.21 | 7.52 | |
| permanent | | | | | | |
| compression | | | | | | |
| set rate (%) | | | | | | |

A viscosity can reflect a degradation degree of polyurethane to some extent. According to the comparison of physical properties of the recovered products in Example 4 (ZnA₂) and Comparative Examples 1 to 4 (as shown in Table 3), the degradation system including the dual-anion catalyst ZnA₂ can effectively reduce the viscosity and remarkably boost the degradation degree. It indicates that a catalytic effect of the dual-anion catalyst based on a synergistic effect is far superior to a catalytic effect of any individual component alone. Similarly, performance data of the regenerated foams in Table 5 leads to the same conclusion: A degradation product obtained when the dual-anion catalyst ZnA₂ is added has a much better quality than degradation products of the comparative examples. When added at 30%, the recovered product can also improve the mechanical properties of a regenerated foam. Therefore, the present disclosure holds significant implications for the degradation of polyurethane and even carbonyl-containing polymers, and is of great importance for the further industrial-scale recycling of waste polyurethane.

The above are merely preferred embodiments of the present disclosure, and are not intended to limit the present disclosure in any way. Although the present disclosure has been disclosed above through the preferred embodiments, these embodiments are not intended to limit the present disclosure. Any person skilled in the art may make some changes or modifications using the technical content disclosed above without departing from the scope of the technical solutions of the present disclosure to obtain equivalent embodiments with comparable effects. Any simple modification, equivalent change, and modification made to the above embodiments according to the technical essence of the present disclosure without departing from the content of the technical solutions of the present disclosure shall fall within the scope of the technical solutions of the present disclosure.

What is claimed is:

1. A preparation method of a dual-anion catalyst, comprising following steps:
1) dissolving a metal salt in deionized water to produce a metal salt solution;
2) adding a nitrogen-containing heterocyclic compound with hydroxyl or amino as a substrate to a reactor, adding the metal salt solution dropwise to the reactor, and conducting condensation reflux at 60° C. to 150° C. for 1 h to 8 h;
3) after the condensation reflux in the step 2) is completed, adding an amine compound dropwise to the reactor, and conducting a reaction for 2 h to 5 h; and after the reaction is completed, conducting vacuum filtration to produce a filtrate;
4) subjecting the filtrate obtained in the step 3) to evaporation; and
5) oven-drying a product obtained in the step 4) to produce the dual-anion catalyst.

2. The preparation method of the dual-anion catalyst according to claim 1, wherein in the step 1), the metal salt is one or more of zinc acetate, magnesium acetate, zinc nitrate, cadmium nitrate, nickel nitrate, and cobalt nitrate.

3. The preparation method of the dual-anion catalyst according to claim 1, wherein in the step 2), the nitrogen-containing heterocyclic compound with hydroxyl or amino is one or more of 4-(hydroxymethyl)imidazole, 2-hydroxymethyl-1-methylimidazole, 6-(hydroxymethyl)pyridin-3-ol, 3,4-bis(hydroxymethyl)furan, and 6-hydroxymethylquinoline.

4. The preparation method of the dual-anion catalyst according to claim 1, wherein in the step 2), the metal salt solution is slowly added dropwise to the reactor for 20 min to 40 min.

5. The preparation method of the dual-anion catalyst according to claim 1, wherein in the step 2), the condensation reflux is conducted at 100° C. to 150° C. for 1 h to 4 h.

6. The preparation method of the dual-anion catalyst according to claim 1, wherein in the step 3), the amine compound is one or more of phenylethylamine, triphenylguanidine, tetramethylguanidine, and sulfaguanidine.

7. The preparation method of the dual-anion catalyst according to claim 1, wherein in the step 3), after the condensation reflux in the step 2) is completed, the amine compound is added dropwise to the reactor, and then the reaction is conducted for 2 h to 3 h.

8. The preparation method of the dual-anion catalyst according to claim 1, wherein a molar ratio of the nitrogen-containing heterocyclic compound with hydroxyl or amino, the metal salt, and the amine compound is (0.3-1):(0.3-1.5):(0.5-1).

9. The preparation method of the dual-anion catalyst according to claim 1, wherein in the step 4), the filtrate obtained in the step 3) is treated for 1 h to 2 h in a rotary evaporator at 50° C. to 60° C.; and
in the step 5), the product obtained in the step 4) is oven-dried for 12 h to 24 h in a forced air oven at 80° C. to 90° C.

10. A use of a dual-anion catalyst prepared by the preparation method according to claim 1 in catalytic degradation of a polyurethane material.

11. The use according to claim 10, wherein in the step 1) of the preparation method, the metal salt is one or more of zinc acetate, magnesium acetate, zinc nitrate, cadmium nitrate, nickel nitrate, and cobalt nitrate.

12. The use according to claim 10, wherein in the step 2) of the preparation method, the nitrogen-containing heterocyclic compound with hydroxyl or amino is one or more of 4-(hydroxymethyl)imidazole, 2-hydroxymethyl-1-methylimidazole, 6-(hydroxymethyl)pyridin-3-ol, 3,4-bis(hydroxymethyl)furan, and 6-hydroxymethylquinoline.

13. The use according to claim 10, wherein in the step 2) of the preparation method, the metal salt solution is slowly added dropwise to the reactor for 20 min to 40 min.

14. The use according to claim 10, wherein in the step 2) of the preparation method, the condensation reflux is conducted at 100° C. to 150° C. for 1 h to 4 h.

15. The use according to claim 10, wherein in the step 3) of the preparation method, the amine compound is one or more of phenylethylamine, triphenylguanidine, tetramethylguanidine, and sulfaguanidine.

16. The use according to claim 10, wherein in the step 3) of the preparation method, after the condensation reflux in the step 2) is completed, the amine compound is added dropwise to the reactor, and then the reaction is conducted for 2 h to 3 h.

17. The use according to claim 10, wherein in the preparation method, a molar ratio of the nitrogen-containing heterocyclic compound with hydroxyl or amino, the metal salt, and the amine compound is (0.3-1):(0.3-1.5):(0.5-1).

18. The use according to claim 10, wherein in the step 4) of the preparation method, the filtrate obtained in the step 3) is treated for 1 h to 2 h in a rotary evaporator at 50° C. to 60° C.; and
in the step 5), the product obtained in the step 4) is oven-dried for 12 h to 24 h in a forced air oven at 80° C. to 90° C.
